(19) United States
(12) Patent Application Publication
(10) Pub. No.: US 2009/0306606 A1
(43) Pub. Date: Dec. 10, 2009

(54) CATHETER HUB ASSEMBLY WITH VASCULAR ACCESS PORT

(75) Inventors: Carol L. Lancette, Hudson Falls, NY (US); Michael S. Zanoni, Glens Falls, NY (US)

Correspondence Address: ANGIODYNAMICS, INC., 603 QUEENSBURY AVENUE, QUEENSBURY, NY, 12804, US

(73) Assignee: AngioDynamics, Inc, Queensbury, NY (US)

(21) Appl. No.: 12/136,149

(22) Filed: Jun. 10, 2008

(51) Int. Cl. A61M 31/00

(52) U.S. Cl. 604/288.02; 604/513

(57) ABSTRACT

A vascular access catheter having a catheter hub assembly and a catheter shaft having a plurality of lumens is disclosed. The hub assembly has a body with an interior cavity and an exterior surface with a plurality of openings that are configured to receive the catheter shaft and a plurality of extension tubes, which are fluidly connected to lumens of the catheter shaft. A vascular access port having a housing, at least one reservoir and a self-sealing septum having an outer surface, is positioned partially therein the interior cavity of the body. A portion of the housing defines a reservoir outlet stem that is in fluid communication with the reservoir. At least a portion of the outer surface of the septum is defined therein a port opening that is defined therein the body and is circumferentially surrounded by portions of the exterior surface of the hub body.

### FIELD OF THE INVENTION

The present invention pertains to the field of medical devices. More particularly, the present invention relates to a catheter hub and a vascular access catheter with a catheter hub assembly and a method of using such vascular access catheter.

### BACKGROUND

Vascular access procedures may be used when a patient needs intravenous antibiotic treatment chemotherapy or anticancer drugs, pheresis, drug infusion, blood sampling, long-term intravenous (IV) feeding for nutritional support, bolus injections, or hemodialysis, for example. Such procedures may involve the insertion of a catheter into a patients blood vessel to provide a method of drawing blood or delivering drugs and nutrients into a patients bloodstream over a period of weeks, months or even years.

Devices used during vascular access procedures may include catheters or subcutaneously implantable ports. Catheters used during vascular access procedures may include peripherally inserted central catheters (PICCs) or hemodialysis catheters, among others. Peripherally inserted central catheter (PICC) lines are inserted into a large vein in the arm and extend into the largest vein (superior vena cava) near the heart and typically provide central IV access for several weeks, but may remain in place for several months. Hemodialysis catheters are used to remove waste products such as potassium and urea from the blood, such as in the case of renal failure. During hemodialysis, waste products that have accumulated in the blood because of kidney failure are transferred via mass transfer from the blood across a semi permeable dialysis membrane to a balanced salt solution. One type of hemodialysis catheter that is well-known in the art is a triple-lumen hemodialysis catheter.

Triple lumen hemodialysis catheters are designed to provide long-term access to the venous system for dialysis and typically have an inflow lumen for withdrawing blood to be treated from a blood vessel and an outflow lumen for returning cleansed blood to the vessel. The distal segment of the catheter is preferably positioned at the junction of the superior vena cava and right atrium to obtain a blood flow of sufficient volume to accommodate dialysis treatment requirements. This allows blood to be simultaneously withdrawn from one lumen, to flow into the dialysis circuit, and be returned via the other lumen. Triple lumen hemodialysis catheters have an additional third lumen that may be used for guidewire insertion, administration and withdrawal of fluids such as antibiotics, chemotherapeutics or other drugs, blood sampling, hydration, or parenteral nutrition, to name a few.

While triple lumen dialysis catheters have many advantages, such catheters may also have disadvantages, such as drug or other fluid leakage from such catheters or the extension tubes when such catheters are used for chemotherapy or high pressure CT injections. If chemotherapy drugs are leaked into the surrounding tissues, this can cause damage, which may be very dangerous and difficult to treat. Further, if certain drugs are administered into a vein in the arm of patients who need frequent injections and intravenous treatments, it is possible to run out of usable veins in which to inject the drugs.

Typically, in triple lumen catheters, three extension tubes extend out of the catheter bifurcate while being in fluid communication with three catheter lumens. This design causes the proximal end of such catheters to be bulky at the insertion site, which can be cumbersome for medical personnel to use. This can also cause the proximal end of the catheter to be heavier compared to typical dual lumen catheters. The extra weight can bear down on the patient during prolonged treatment times and can cause discomfort to the patient, kinking, and an increased risk of infection. The crowding and/or abrasion of the extension tubes and/or clamps may also potentially damage the catheter. The third lumen in such triple lumen catheters is typically infrequently used. This lack of use can cause potential leaking if the extension tube that is connected to the third lumen is left unclamped. The unclamped extension tube may also be prone to infection and may be subject to failure over a long period of time.

As an alternative to vascular access catheters, implantable vascular access ports are also frequently used to overcome problems associated with limited peripheral access and to address the need for frequent venipuncture in patients receiving long-term, intensive therapy. Unlike catheters, which exit from the skin, implantable ports are placed completely below the skin. Ports may be implanted for short or long terms in a patient. Ports are used mostly to treat hematology and oncology patients, but have also been adapted for hemodialysis patients. Ports may also be connected to and used in conjunction with catheters.

A port typically has a reservoir and a self-sealing silicone septum that overlays the reservoir and can be punctured many times with a needle before the need to be replaced. Drugs can be injected into the needle, or blood samples can be withdrawn. The port device is surgically inserted under the skin in the upper chest or in the arm and appears as a bump under the skin. Implanting a subcutaneous port generally requires multiple incisions. In addition to a first incision to place the port, the patient must undergo a second surgical procedure to have the port removed after treatment is completed. To implant a port underneath the skin, a port implantation site is selected and a small subcutaneous pocket is created. A catheter is tunneled from the port site to the blood vessel which is to be catheterized, and the catheter is placed into the blood vessel of interest. The port reservoir is placed under the skin following a small skin incision, connected to the catheter, and then sutured to the surrounding underlying tissue. To administer treatment or to withdraw blood, a medical practitioner must locate the septum underneath the skin and disinfect the area. The port is then accessed by puncturing the overlying skin with a 90° non-coring Huber point needle into the reservoir of the port, although other types of needles may also be used.

As with triple lumen catheters, implantable vascular access ports have many advantages, but they also have several disadvantages. For instance, pneumothorax, or collapse of the lung because of injury to the lung from an inserted needle could occur. Other complications could include hemothorax, or bleeding into the chest because of injury to the blood vessels from the needle at insertion, cellulitis, infection of the skin around the port, and pain or discomfort to the patient when using a needle to inject into the port through the patient's skin, swelling, fever, sepsis, tachycardia, tachypnoea, hypotension, occlusions, thrombus formation, or increased procedure time due to having to replace the port if the port malfunctions or is damaged. The longer the port is left implanted in the patent, the greater the chance the port may become infected. The port may also be prone to rotation or flipping within the patient, which may make it difficult to locate the port and may require the port to be removed and re-positioned within the patient's body.

If used in conjunction, or in addition to, a catheter, implantable ports can also cause an increase in procedure time, due to the extra steps required to implant the port and connect the port to a catheter. These connection steps can also increase procedure costs, the chance of infection, and patient discomfort. The additional need for consideration by medical personnel and patients regarding what device to use, i.e., whether to use a port or a catheter, in cases where the devices are used separately, can also cause an increase in overall procedure time, which can adversely affect the quality of patient care.

A vascular access catheter has not yet been proposed that solves all of the above-mentioned problems. A catheter hub or bifurcate assembly that incorporates a vascular access port within the hub, for use with a vascular access catheter, such as those described above, is provided. The hub assembly has one or two extension tubes, instead of three, for use with a dual or triple lumen vascular access catheter, thereby minimizing the proximal end bulk of the catheter and decreasing the chance of infection and patient discomfort.

A catheter hub for use with a vascular access catheter, such as dual lumen or triple lumen catheter, is provided. The catheter hub assembly incorporates a vascular access port that is fluidly connected to a third lumen of the catheter that is capable of being used for injections or infusion of drug treatments, such as in chemotherapy or for high pressure CT injections, or urokinase, to effectively break up fibrin sheath buildup or other occlusive material along the catheter shaft.

A vascular access catheter with a catheter hub assembly is also provided. This catheter hub assembly incorporates a vascular access port that is fluidly connected to third lumen of the vascular access catheter shaft and can be used for injections or infusion of drugs or high pressure CT injections.

A further purpose is to provide a vascular access catheter, such as a hemodialysis catheter or a peripherally inserted central catheter (PICC), with a catheter hub assembly having a vascular access port positioned partially within the catheter hub, thereby combining two products into one, providing multiple functionalities, eliminating the need for multiple incisions, thereby shortening procedure time, and decreasing costs and patient discomfort.

Various other purposes and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description.

### SUMMARY

A catheter hub is provided for use with a vascular access catheter. The catheter hub has a body that has a proximal end, a distal end, an exterior surface, and defining an interior cavity having a bottom surface. The exterior surface of the body has a plurality of openings. At least one opening of the plurality of openings is defined in the proximal end, and at least one opening of the plurality of openings is defined in the distal end. The catheter hub also has a vascular access port that is positioned at least partially therein the interior cavity. The vascular access port has a housing that has a base and a wall extending therefrom the base, and at least one self-sealing septum having an outer surface that is configured for a leak-proof connection to an upper portion of the wall of the housing. A portion of a bottom surface of the at least one septum and portions of the wall and base of the housing define at least one reservoir. A portion of the housing is in fluid communication with the at least one reservoir, and at least a portion of the outer surface of the at least one septum is defined within a port opening of the plurality of openings of the body.

In another aspect, a vascular access catheter that has a catheter hub assembly is provided. The catheter hub assembly of the catheter has a body having a proximal end, a distal end, an exterior surface, and an interior cavity having a bottom surface. The exterior surface of the body further defines at least one proximal opening defined in the proximal end of the body. The exterior surface of the body further defines at least one distal opening defined in the distal end of the body that is configured to receive at least a portion of a proximal portion of the catheter shaft. The body further defines at least one channel that is integral to the at least one distal opening. The hub assembly has a plurality of extension tubes, each extension tube having a proximal end and a distal end. Each of the proximal openings is configured to receive the distal end of one extension tube, which tubes are fluidly connected to a proximal portion of one channel of the body. The hub assembly also has a vascular access port that is positioned at least partially in the interior cavity of the hub. The vascular access port has a housing having a base and a wall extending therefrom the base and at least one self-sealing septum having an outer surface. The septum is mounted in an upper portion of the wall of the housing. A portion of a bottom surface of the septum and portions of the wall and base of the housing define the reservoir. A portion of the housing is in fluid communication with the at least one reservoir. At least a portion of the outer surface of the septum is defined in a port opening that is defined in the body.

In a further aspect, a method of infusing an infusate into patient body through the vascular access catheter with catheter hub assembly is also provided.

### BRIEF DESCRIPTION OF THE SEVERAL VIEWS OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a partial sectional view of a vascular access catheter shaft with a catheter hub assembly.

FIG. 1B illustrates cross-sectional views of the hub assembly of FIG. 1A along lines A-A, B-B, C-C, and D-D.

FIG. 2A illustrates a partial sectional view of an additional embodiment of the vascular access catheter shaft and catheter hub assembly.

FIG. 2B illustrates cross-sectional views of the vascular access catheter shaft and catheter hub assembly of FIG. 2A, along lines H-H, J-J, and K-K.

FIG. 3A illustrates a sectional view of the bottom portion of the snap-fit catheter hub body.

FIG. 3B illustrates a plan view of the top portion of the snap-fit catheter hub body.

FIG. 3C illustrates a side plan view of the catheter hub of FIGS. 3A and 3B.

FIG. 3D illustrates a sectional view of one embodiment of the bottom portion of the snap-fit catheter hub of the catheter hub assembly with extension tubes.

FIG. 4A is a side plan view of the vascular access port of the hub assembly connected to an extension tube.

FIG. 4B is a top plan view of the vascular access port of the hub assembly of FIG. 4A.

FIG. 5A is a side plan view of an additional embodiment of the vascular access port of the catheter hub assembly.

FIG. 5B is a front plan view of the vascular access port of FIG. 5A.

FIG. 5C is a top plan view of an additional embodiment of the vascular access port of FIGS. 5A and 5B connected to an extension tube.

FIG. 6 is a sectional view of the catheter hub assembly with an additional embodiment of the vascular access port in the catheter hub with a needle.

### DETAILED DESCRIPTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention.

The skilled artisan will readily appreciate that the devices and methods described herein are merely exemplary and that variations can be made without departing from the spirit and scope of the invention. It is also to be understood that the terminology used herein is for the purpose of describing particular embodiments only and is not intended to be limiting.

Ranges may be expressed herein as from “about” to one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independents of the other endpoint. As used herein, the term “proximal” means closer to the operator while the term “distal” means further away from the operator than proximal.

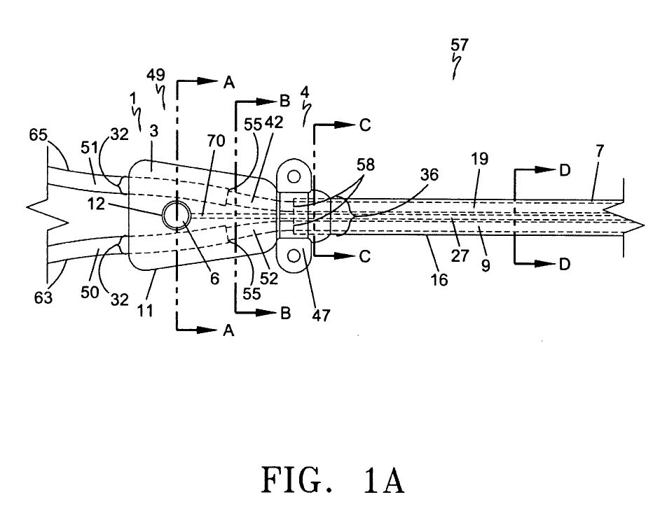

Referring now in detail to the drawings, in which like reference numerals indicate like parts or elements throughout the several views, in various embodiments, and referring to FIGS. 1-6, presented herein is an exemplary catheter hub 49 and a vascular access catheter, such as a hemodialysis catheter or peripherally inserted central catheter (PICC), having a catheter hub assembly, and a method of injecting infusates or contrast agents under high pressure CT injections into the vascular access catheter through the hub assembly. In another aspect, the vascular access port of the present invention may be used with any suitable dual or triple lumen catheter.

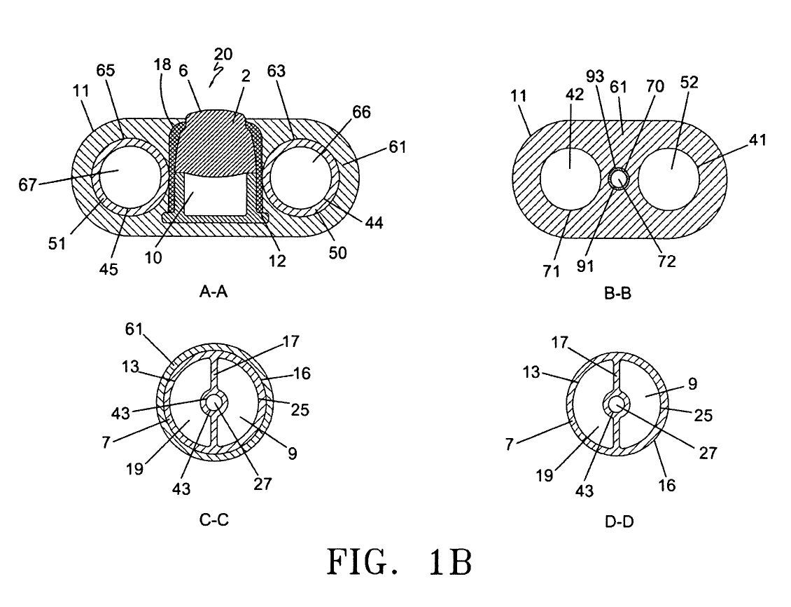

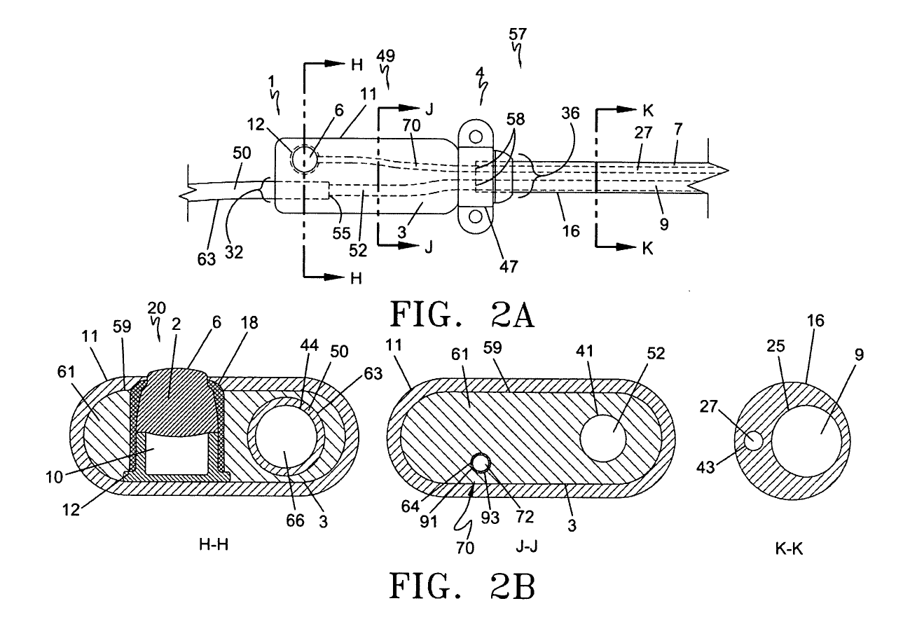

In one exemplary aspect, FIGS. 1A through 2B illustrate one embodiment of the catheter hub assembly 57. The catheter hub assembly 57 has a catheter hub 49. The catheter hub 49 has a proximal end 1 and a distal end 4. In this exemplary aspect, the distal end 4 of the catheter hub 49 may comprise at least one fixed or rotatable suture ring 47 coaxially arranged around the distal end 4 of the hub 49. In one aspect, the catheter hub 49 has a body 11. In one exemplary aspect, the catheter hub 49 may be approximately 1.40 inches long. In one exemplary aspect, the width of the hub 49 tapers from a first diameter of approximately 0.75 inches at the widest portion of the proximal end 1 of the hub 49 to a smaller diameter of approximately 0.32 inches at the narrowest portion of the distal end 4.

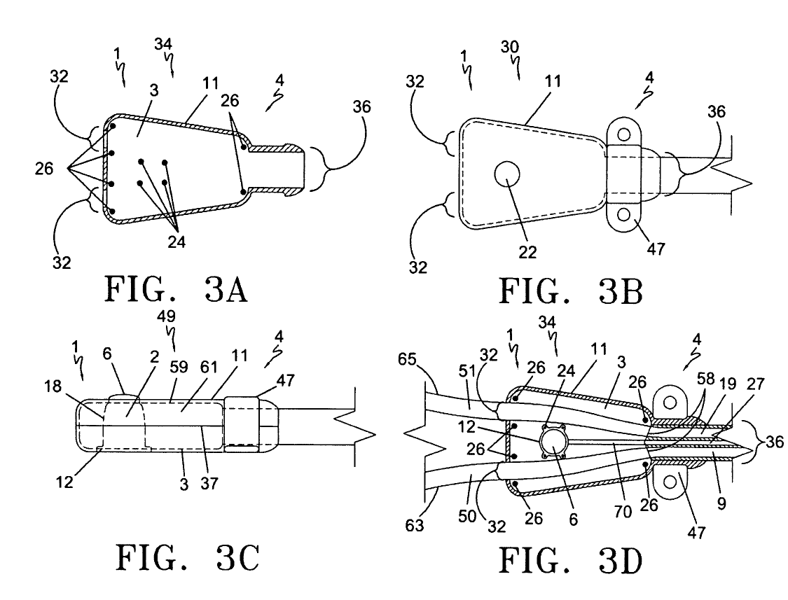

In one exemplary aspect, the catheter hub 49 may be made of any suitable polymeric material, such as, but not limited to, polyurethane, polypropylene, Carbothane, Tecoflex®, Isoplast, polyamide, nylon, polyether block material (PEBA), polyethethersulfone (PES), PP, PE, PCV, ABS resin, or mixtures and copolymers thereof, to name a few. One skilled in the art will recognize that other suitable materials may be used for the hub body 11. In one aspect, the entire hub assembly 57 may be either insert molded, as illustrated in FIGS. 1A-2B or snap-fit together, as illustrated in FIGS. 3A-5C. In the insert-molded hub, the hub body 11 is insert molded over the distal end of a plurality of extension tubes 50, 51, described below and at least a portion of the proximal portion of a catheter shaft 7. In another aspect, the catheter hub assembly 57 may be assembled using a combination of a snap-fit technique followed by a potting technique, which techniques are known in the art and described in more detail below.

In the assembled state, as illustrated in FIGS. 1A through 2B, for example, hub assembly 57 comprises a plurality of extension tubes 50, 51, each having a proximal end and a distal end. FIGS. 1A through 2B illustrate the hub assembly 57 after it has been made using an insert molding procedure. In one aspect, the hub 49 is disposed around at least a portion of the distal portion of at least one of extension tubes 50, 51 at the proximal end 1 of the hub 49 and at least a portion of the proximal portion of a catheter shaft 7 that has an outer wall 16 at the distal end 4 of the catheter hub 49. In one aspect, the exterior surface of the hub body 11 defines a plurality of openings 32, 36. In one aspect, at least one opening 32 of the plurality of openings is defined therein the proximal end 1 of the body 11, and at least one opening 36 of the plurality of openings is defined therein the distal end 4 of the hub body 11, also illustrated in FIGS. 1A, 2A, 3A, 3C, and 3D, for example. In one aspect, each of the proximal openings 32 are configured for receiving the distal end of at least one of extension tube 50, 51. In another aspect, at least one distal opening 36 is configured for receiving at least a portion of a proximal portion of catheter shaft 7. The diameter of openings 32, 36 may be adjusted to accommodate the outer diameters of the outer walls 63, 65 of extension tubes 50, 51, respectively, and the diameter of the outer wall 16 of the catheter shaft 7. Each opening 32, 36 forms an inner rim against which the outer walls 63, 65 of the distal end of extension tubes 50, 51, and the outer wall 16 of the catheter shaft 7, respectively, abut against upon insertion into the respective openings 32, 36. The body 11 also defines at least one channel 42, 52 that is integral to the distal opening 36, as illustrated in FIG. 1A.

In one aspect, in the assembled state, extension tubes 50, 51 extend out of the proximal end 1 of the catheter hub 49 through openings 32 for connection to a dialysis machine. It is contemplated that any of extension tubes 50 or 51 may have an identification (ID) tag denoting the flow rate and/or volume of fluid to be injected into the catheter shaft 7 through the extension tubes 50, 51. Extension tubes 50, 51 may be comprised of polyurethane, as well as other materials, such as, but not limited to Carbothane or Tecoflex®. In one aspect, the extension tubes 50, 51 are composed of Carbothane. The proximal portion of the catheter shaft 7 extends distally from the hub 49 through opening 36 toward the distal end of the catheter (partially illustrated).

In one exemplary aspect, opening 36 may be sized to receive a catheter shaft 7 with an outer wall 16 having an outer diameter of approximately 0.203 inches, although, as one skilled in the art will appreciate, other diameter catheters are within the scope of this invention. Catheters for use with this catheter hub assembly 57 may also be, but are not limited to, a 15.5 Fr catheter, although, as one skilled in the art will appreciate, any suitable size catheter may be used. In one aspect, the catheter hub assembly 57 may be used with catheters that have a unitary catheter shaft 7. In one aspect, the catheter shaft 7 may be composed of, but is not limited to, carbothane, Tecoflex®, silicone, polyurethane, polyethylene, and teflon, or any suitable polymeric material. In another aspect, the catheter shaft 7 may also contain a radiopaque material to enhance visibility under fluoroscopy. The catheter shaft 7 may optionally be comprised of materials of different durometers to produce a shaft 7 with enhanced flexibility.

In one aspect, as illustrated in FIGS. 1A through 2B, for example, extension tube 50 is defined by outer wall 63 and inner wall 44, and lumen 66, through which infusates or other fluids, such as blood, may travel through. Similarly, extension tube 51 is defined by outer wall 65, inner wall 45, and lumen 67, through which infusates or other fluids, such as blood, may travel. As also illustrated in FIG. 1B, in one exemplary aspect, the hub body 49 defines an interior cavity 61 having a bottom surface 3 which is part of an interior surface 59 of the interior cavity 61.

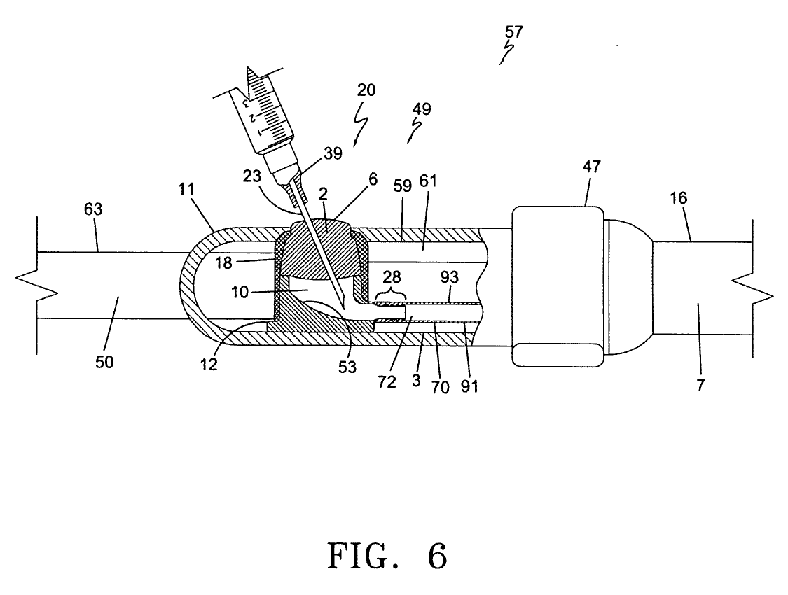

In one aspect, as illustrated in FIG. 1B, the first and second extension tubes 50, 51 are defined in surrounding relationship to a vascular access port 20 that is positioned at least partially therein the interior cavity 61 of the hub body 11. In one exemplary aspect, the vascular access port 20 is positioned between extension tubes 50, 51, respectively. In one aspect the vascular access port 20 comprises a housing 18 having a base 12 and a wall extending therefrom the base 12, at least one reservoir 10, and at least one self-sealing septum 2 having an outer surface 6. In one aspect, the septum 2 of the vascular access port 20 may be configured to receive at least a portion of a needle. In one aspect, the penetrable septum 2 is comprised of a self-resealing polymer, which is preferably an elastomer, such as silicon rubber or a latex, and which is adapted to permit access using a hypodermic needle 23 (illustrated in FIG. 6) into the reservoir 10 formed within the access port 20.

In one aspect, the vascular access port housing 18 may be composed of materials, such as titanium or plastic, silicone rubber, polyurethane, polysulfone, or any suitable combination of these materials. In one aspect, the housing 18 of the port 20 may be of any suitable shape or size, provided that it is configured to fit inside of the catheter hub 49. The vascular access port 20 of the present invention may be MRI-compatible. The base 12 of the housing 18 is positioned therein the interior cavity 61 of the body 11 substantially parallel relative to a horizontal plane bisecting the catheter body 11. In one exemplary aspect, in the assembled state, the base 12 of the housing 18 is mounted therein a portion of the interior cavity 61 of the body 11, as described below. In one aspect, the septum 2 is mounted in an upper portion of the wall of the housing 18. The base 12 is sealingly engaged to a portion of the bottom surface 3 of the interior cavity 61 of the body 11. The catheter hub 49 further comprises a means for securing the base 12 to the bottom surface 3 of the interior cavity 61 of the body 11. Means for securing the base 12 to the bottom surface 3 of the interior cavity 61 of the body 11 may include one or more of a plurality of securement tabs 24, 26 (described below), adhesives, and the like. The size and shape of the port 20 may be adjusted, based on the type of port 20 and catheter that will be used. In one aspect, the port 20 may be a low profile port.

In one aspect, at least a portion of the septum 2 is defined therein the housing 18, such that it forms an interference fit with the housing 18. At least a portion of the outer surface 6 of the septum 2 is thus defined therein a port opening 22 (FIG. 3B) of the plurality of openings of the body 11. In one aspect, at least a portion of the septum 2 is compressed within the housing 18, causing the septum 2 to bulge upward from the housing 18 through a preformed port opening 22 (FIG. 3B) in the exterior surface of the catheter hub 49, as illustrated in FIGS. 1B, 2B, 3C, and 6, for example. At least a portion of the outer surface of the septum 2 that is positioned therein the port opening 22 is circumferentially surrounded by portions of the exterior surface of the hub body 11. In the compressed state, at least a portion 6 of the outer surface of the septum 2 positioned therein the port opening 22 extends outwardly above a plane that bisects the port opening 22. In one aspect, the access port 20 described herein may be identical to those described in U.S. provisional patent application Ser. Nos. 60/970,816 and 60/977,636, incorporated herein by reference.

In one aspect, the vascular access port 20 may have at least one reservoir 10, as illustrated in FIGS. 1B and 2B. The at least one reservoir 10 is defined by a portion of a bottom surface of the at least one septum 2 and portions of the wall and base 12 of the housing 18. In one exemplary aspect, the reservoir may be constructed of a biocompatible material, such as titanium or stainless steel, or any other suitable biocompatible material. The reservoir may also be rounded with rounded edges, or may be made of any suitable shape. In one aspect, the reservoir 10 may have a funnel-like shape with an inner surface 53, as illustrated in FIG. 6. The funnel shape helps to guide inserted needle 23 and/or at least a portion of a guidewire through the reservoir 10 and further into the lumen 72 of the connection tube 70 and into the third lumen 27 of the catheter shaft 7.

In one aspect, as illustrated in FIG. 1A, for example, at least a portion of the plurality of extension tubes 50, 51 extend substantially longitudinally in the interior cavity 61 of the hub 49 relative to a longitudinal axis of the hub body 11. The extension tubes 50, 51 may be slightly curved or angled relative to the longitudinal axis of the hub, or the extension tubes 50, 51 may be substantially straight or parallel relative to the longitudinal axis of the hub, or the extension tubes 50, 51 may be a combination of substantially curved, angled, or straight tubes. At least a portion of the distal end of at least one of extension tubes 50, 51 is fluidly connected with at least a portion of the proximal portion of at least one of channels 42, 52 in the interior cavity 61 of the hub body 11 at transition point 55. As described in FIG. 1A, in one aspect, the distal end of the first extension tube 50 is in fluid communication with the proximal portion of the first channel 52, and the distal end of the second extension tube 51 is in fluid communication with the proximal portion of the second channel 42. Channels 42, 52 are defined within the interior cavity 61 of the hub body 11 in surrounding relationship to the vascular access port 20 and are created by molding pins during the insertion molding process of the hub 49. Channel 42 is defined by an inner wall 71, and channel 52 is defined by an inner wall 41, as illustrated in the cross-sectional views of FIG. 1B. In one aspect, at least a portion of the distal portion of lumen 66 of extension tube 50 is in sealed fluid communication with channel 52, and lumen 67 of extension tube 51 is in sealed fluid communication with channel 42, illustrated in FIG. 1A and the cross-sectional views of FIG. 1B.

In one aspect, in the assembled state, a vascular access catheter comprising the catheter hub assembly 57 may have a catheter shaft 7 that comprises a plurality of lumens. The plurality of lumens may comprise a first lumen 9 and a second lumen 19, illustrated in FIGS. 1A and 1B. Lumens 9 and 19 extend longitudinally through substantially the entire length of the catheter shaft 7 from the distal most end of the catheter (partially illustrated) into the cavity 61 of the catheter hub 49.

In one aspect, as illustrated in FIGS. 1A and 1B, the outer wall 16 of the catheter shaft 7 surrounds the outflow lumen 9 and inflow lumen 19, which are separated by and share a common internal septum 17 defined therein the catheter shaft 7. In one exemplary aspect, the internal septum 17 can have a width of approximately 0.144 inches. In one aspect, the inflow lumen 19 can have a D-shaped lumen configuration or a substantially D-shaped lumen configuration, as illustrated in FIG. 1B, and the outflow lumen 9 can also have a D-shaped lumen configuration or a substantially D-shaped lumen configuration, as illustrated in FIG. 1B. In one exemplary embodiment, the height of each substantially double-D lumen may be approximately 0.064 inches. Of course, it is contemplated that the lumens of the catheter shaft 7 may have any suitable cross-sectional lumen shape as required for the particular use of the catheter shaft 7.

In one aspect, outer wall 16 and inner wall 13 define the inflow lumen 19. In another aspect, the outflow lumen 9 is defined by outer wall 16 and inner wall 25. The effective cross-sectional area of each lumen 19 and 9 may be substantially equalized, which aids in providing substantially equalized flow rates in both the inflow and outflow directions. In one non-limiting example, the resulting cross-sectional area of each respective lumen 19 and 9 is approximately 0.0065 inches². The inflow lumen 19 can be exemplarily used for withdrawal of blood from the patient during dialysis or other procedures. In another aspect, the outflow lumen 9 can be exemplarily used for delivering cleansed blood back into the patient's circulatory system. One skilled in the art will appreciate that, although designated herein as inflow and outflow lumens, dialysis may be performed by reversing the blood flow through the lumens. Hence, the terms first lumen and second lumen may also be used herein to designate the interchangeability of the outflow and inflow lumens, respectively. In one exemplary aspect, blood can be withdrawn from the vessel of the patent into lumen 19 where it is passed through the extension tube 51 and into the dialysis machine. Blood can be returned to the patient through extension tube 50 into lumen 9, into the vessel of a patient.

In one aspect, the distal portion of at least one channel 42, 52 is fluidly connected to the proximal portion of the plurality of lumens of the catheter shaft 7 at the distal end of the body 4. In one aspect, channel 42 is in sealed fluid communication with at least a portion of lumen 19 of the catheter shaft 7 and is fluidly connected to lumen 19 of the catheter shaft 7 at transition point 58. Channel 52 is fluidly connected to at least a portion of lumen 9 of the catheter shaft 7 at transition point 58 and is in sealed fluid communication with lumen 9. Alternatively, in one aspect, lumens 19, 9 may be in sealed fluid communication with transition tubing (not illustrated), which in turn, may be fluidly sealed to channels 42, 52. At transition point 58 in the insert-molded hub 49, channels 42, 52 form a seamless transition with the lumens of the catheter shaft 7, while in the snap-fit hub 49, the channels 42, 52 are sealingly engaged or welded together with lumens 9, 19 of the catheter shaft 7 at transition point 58.

In one aspect, the vascular access catheter shaft 7 described herein further comprises a third lumen 27 or infusion lumen 27, as illustrated in FIGS. 1A through 2B, for example. The terms “third lumen 27” and “infusion lumen 27” are used interchangeably herein to denote the fact that the third lumen 27 can also be used as an infusion lumen in a dual lumen catheter shaft configuration (see FIG. 2A). In one aspect, lumen 27 has an inner wall 43, as illustrated in FIG. 1B. In another exemplary aspect, third lumen 27 can have a generally smaller transverse cross-sectional area than the transverse cross-sectional area of lumens 19 and 9.

In one exemplary aspect, as illustrated in FIGS. 1A and 2A, the distal end of connection tube 70 is fluidly connected to at least a portion of the proximal end of the catheter shaft 7 and is in fluid communication with the third lumen 27 of the catheter shaft 7. The distal end of the connection tube 70 is fluidly connected to lumen 27 at transition point 58 in the hub cavity 61. The connection tube 70 is defined by an outer wall 93 and inner wall 91, and a lumen 72. A cross-sectional view of the connection tube 70 is illustrated in FIG. 1B at line B-B. The proximal end of the connection tube 70 is fluidly connected to vascular access port 20, as described below and also illustrated in FIGS. 1A and 2A, for example. The length of the connection tube 70 between the vascular access port 20 and the proximal portion of the catheter shaft 7 may be any suitable length, such that the vascular access port 20 may be positioned at variable lengths from the distal end 4 of the hub 49. In one aspect, connection tube 70 is defined within the interior cavity 61 of the catheter hub 49 between the outflow lumen 9 and the inflow lumen 19, as illustrated in FIGS. 1A, 1B, and 3D. In other exemplary aspects, the connection tube 70 may be positioned therein the interior cavity 61 of the hub 49 on either side of the extension tubes 50, 51.

In one exemplary aspect, and not meant to be limiting, the inner diameter of third lumen 27 may be approximately 0.037 inches. In one aspect, the third lumen 27 is in selective fluid communication with an infusate. Thus, third infusion lumen 27 may be used for the injection and delivery of drugs, such as, for example and without limitation, urokinase or other anti-thrombotic agents, fluids, such as contrast media, or blood sampling, perfusion, infusion, plasmapheresis, chemotherapy, high pressure CT injections, and the like. Infusates, such as drugs, may be injected through a needle 23 (FIG. 6) into the septum 2 through the outer surface 6 of the septum of the vascular access port 20 that is embedded in the hub 49 into reservoir 10, and into the third lumen 27, and into a patients body. Infusates may also be injected to dissolve fibrin sheaths or other occlusive material that has the tendency to form along the catheter shaft 7 after the catheter has been implanted in a patient body. Various fibrinolytic enzymes may also be used to reduce the aggregation of fibrin and thrombi, such as tissue plasminogen activator (tPA), urokinase, streptokinase, and other related compounds. Other solutions, such as heparin, may be inserted through the port 20 and into the reservoir 10 and the catheter shaft 7 to prevent blood coagulation. In this aspect, the need for the practitioner to place a secondary vascular access device for periodic sampling and infusion is eliminated.

In one exemplary alternative embodiment, and as illustrated in FIG. 2B, the connection tube 70 and/or the third/infusion lumen 27 may have a liner 64 that is positioned thereon at least a portion of the inner wall 91 of the connection tube 70 or at least a portion of the inner wall 43 of the third lumen 27. In one aspect, liner 64 may be positioned thereon at least a portion of the inner wall of the third lumen 27, as described above (illustrated in FIG. 6). In this aspect, the liner 64 can be a tubular structure that functions to increase the burst pressure of the third lumen 27. Burst pressure is defined herein as the amount of pressure that the lumen 27 may withstand during high pressure applications, such as contrast media injections, before rupturing when infusates, such as contrast agents, are injected into the catheter shaft 7 under high pressure. In this aspect, the liner 64 can be formed of a liner material with a higher yield stress than the material of the catheter shaft 7.

In various aspects, the liner 64 can protect the inner wall 43 of the lumen 27 from erosion due to drug and chemical use, thereby allowing the catheter hub assembly 57 and the catheter shaft 7 to be more resistant to drug therapy, and also supports high pressures for the purpose of CT injection, thereby allowing the catheter hub assembly 57 and catheter to be effectively used for high pressure CT injection, and eliminating the need for port placement. As noted above, the liner 64 may be made of any suitable material that may increase the burst pressure of the lumen 27, such as, but not limited to, nylon, polyamide, and the like. The liner 64 can also function to reduce friction over a guidewire. In one example, the liner 64 may have a wall thickness of between approximately 0.002 and 0.005 inches. Optionally, the liner 64 can comprise a higher strength material than the catheter shaft 7.

In one aspect, the reservoir 10 of the access port 20 is fluidly connected to the third lumen 27 of the catheter shaft 7 of the catheter hub assembly 57 via the extension tube 70. In one aspect, the third lumen 27 extends proximally into the catheter hub cavity 61. In one exemplary embodiment, at least a portion of the third lumen 27 of the catheter shaft 7 is positioned therein the interior cavity 61 of the hub body 11 between the first lumen 9 and the second lumen 19 of the catheter shaft 7. In one aspect, the third lumen 27 extends substantially the entire length of the catheter shaft 7, and the transverse cross-sectional area of the third lumen 27 is substantially less than the cross-sectional area of the lumens 19 and 9 of the catheter. Third lumen 27 is in selective communication with an infusate and can be used for the injection of infusates such as medication or fluids, including glucose or saline solutions through septum 2 of the port 20, into the patient's body. In one aspect, the third lumen 27 is also configured to selectively receive at least a portion of a guidewire.

In one aspect, a dual lumen catheter shaft 7, as illustrated in FIGS. 2A and 2B, can be used with the catheter hub assembly 57. In this embodiment the catheter hub assembly 57 is comprised of a catheter hub 49 that has a rectangular shape. In various alternative embodiments, either or both of the insert molded or the snap-fit catheter hub assembly 57 and the port 20/septum 2 may have any suitable shape or size, such as, but not limited, to triangular, rectangular, oval, and the like. In the embodiment described in FIGS. 2A and 2B, the port 20 is secured to the bottom surface 3 of the interior cavity 61 of the hub 49 prior to assembly of the catheter hub 49, as described previously. At least a portion of the outer surface of the septum 2 that is positioned therein the port opening 22 (FIG. 3B) is circumferentially surrounded by portions of the exterior surface of the hub body 11. In the compressed state, at least a portion 6 of the outer surface of the septum 2 positioned therein the port opening 22 extends outwardly above a plane that bisects the port opening 22, as described in the previous embodiments illustrated in FIGS. 1A and 1B.

In this aspect, at least a portion of the distal portion of the lumen 66 of extension tube 50 is in sealed, fluid communication with channel 52 in the interior cavity 61 of the hub body 11, being connected to channel 52 at transition point 55. Channel 52 is in sealed fluid communication with lumen 9 of the catheter shaft 7 and is fluidly connected with lumen 9 at transition point 58 at the distal end 4 of the hub body 11. The catheter shaft 7 with outer wall 16 extends distally from opening 36 at the distal end 4 of the hub 49.

In one aspect, the infusion lumen 27, which extends substantially the entire length of the catheter shaft 7 is fluidly connected with connection tube 70 at transition point 58, allowing lumen 72 of connection tube 70 to be in sealed, fluid communication with lumen 27 of the catheter shaft 7. As described above, the proximal end of connection tube 70 is in sealed, fluid communication with the at least one reservoir 10 of the port 20 after the port is secured to the bottom surface of the interior cavity 61, prior to completion of the hub assembly 57. The plurality of extension tubes 50, 51, at least a portion of channels 42, 52 and at least a portion of the proximal portion of the catheter shaft 7 extend substantially longitudinally in the interior cavity 61 of the hub relative to a longitudinal axis of the hub body 11.

In one exemplary aspect, as illustrated in FIG. 1B, the port 20 which is fluidly connected to connection tube 70, is positioned adjacent to the extension tube 50/channel 52 within the cavity 61 of the hub body 11. In one aspect, the port 20 may be positioned on either side of the extension tube 50/channel 52 in the interior cavity 61 of the hub 49. As illustrated along cross-section D-D, lumen 9 of the catheter shaft 7 is defined by outer wall 16 and inner wall 25, and is positioned adjacent to the third/infusion lumen 27 in the catheter shaft 7.

The catheter hub 49 of this invention is beneficial because it provides an automatic third lumen 27 that is available to be used for such purposes as, but is not limited to, drug infusion, CT injections, and blood sampling, to name a few. In another aspect, the third lumen 27 may be heparin-locked in the same manner that other catheter lumens of the catheter may be heparin-locked. This is critical because the third lumen 27 is small in diameter, so it is important to decrease the chance that the third lumen 27 will become occluded.

The catheter hub assembly 57 described herein is also beneficial because it only requires one or two extension tubes 50, 51, rather than three, as in typical triple lumen hemodialysis catheters, and thereby eliminates at least one extension tube that would otherwise be connected to a third infrequently used lumen. The elimination of the third extension tube is beneficial because it allows a user to perform fewer procedural steps when opening or closing the extension tubes. The elimination of the third extension tube also decreases the possibility of a user confusing or mixing up the extension tubes.

The catheter hub design described herein is also beneficial because it prevents leaks or malfunctions from occurring in the catheter extension tubes or the catheter. This helps to prevent potential leaking of the extension tube if the extension tube that is connected to the third lumen is left unclamped and helps to prevent the catheter shaft from breaking. The chance of infection and of catheter failure over a long period of time is also decreased with the catheter hub design disclosed herein, thereby reducing the need for a catheter repair kit.

Removing the third leg of a typical triple lumen dialysis catheter and replacing it with a self-healing port in the bifurcate removes proximal end bulk and excess extension tubes extending from the bifurcate and decreases treatment and procedure time. The catheter hub 49 provided herein also allows for a lower profile of the hub 49, decreases excess weight and lessens the chance of infection, thereby increasing patient comfort and the quality of medical care.

The catheter hub assembly 57 described herein is beneficial because it combines several vascular access products into one, i.e., a vascular access port and a hemodialysis catheter, such as that illustrated in FIGS. 1A-1B. In an alternative aspect, the catheter hub assembly 57 may combine the functionalities of a vascular access port and a peripherally inserted central catheter (PICC) into one product, such as that illustrated in FIGS. 2A and 2B. This provides a more cost-effective medical device, which allows a medical practitioner to utilize only one vascular access site in a patient instead of multiple access sites, thereby enabling several medical procedures to be performed at simultaneous or different times. For instance, a patient can simultaneously undergo hemodialysis and blood sampling, or other procedures for which a vascular access port may be used. In another aspect, the catheter hub assembly 57 may be used with a catheter and a vascular access port such that several different procedures may be performed at different times using the catheter hub assembly 57, such as, for example, hemodialysis or high pressure CT injections.

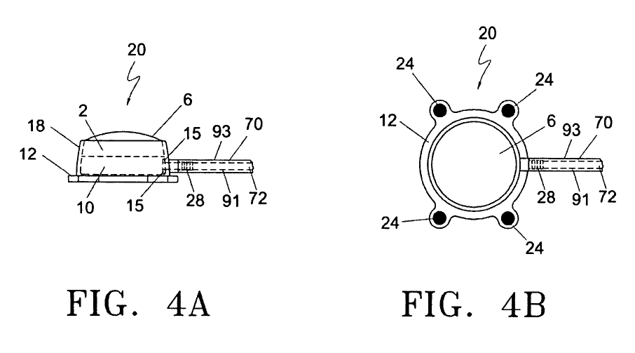

The catheter hub assembly 57 may be assembled using several different techniques. In one aspect, the entire hub assembly 57 may be insert molded. Before the catheter hub assembly 57 is insert molded, the port 20 is assembled with septum 2. The proximal end of the connection tube 70 is fluidly connected to the assembled port 20 and is attached or molded to the port body 11 at reservoir outlet stem 28 in the interior cavity 61 of the hub 49, described, for example, in FIGS. 4A-4B. This allows the connection tube 70, and one lumen of the plurality of lumens of the catheter shaft 7 to be in fluid communication with the at least one reservoir 10 of the port 20. In one aspect, the connection tube 70 is in fluid communication with the third lumen 27 of the catheter shaft 7. The reservoir outlet stem 28 is defined by portions of the housing 18 and is in fluid communication with the at least one reservoir 10.

In several exemplary aspects, the stem 28 may comprise a means for securing the third lumen 27 of the catheter shaft 7 to the outlet stem 28. The inner wall 91 of the connection tube 70 can be attached to the stem 28 by placing the connection tube 70 over a barbed fitting, by using a press-fit design, or a solvent bond or molded-on connection, a standard male/female connection, or an adhesive material or a sealant, such as an epoxy, or any other type of suitable connection means. The barbed fitting of the stem 28 may encircle the outlet stem 28 and flare radially outwardly. In one aspect, illustrated in FIG. 4A, the outer surface 93 of the connection tube 70 may be secured to the inner surface 53 of the reservoir 10 using securement tabs 15 or an adhesive or sealant, such as an epoxy. In another aspect, ultrasonic welding or a snap-fit mechanism may be used to connect the third lumen 27 of the catheter shaft 7 to the outlet stem 28.

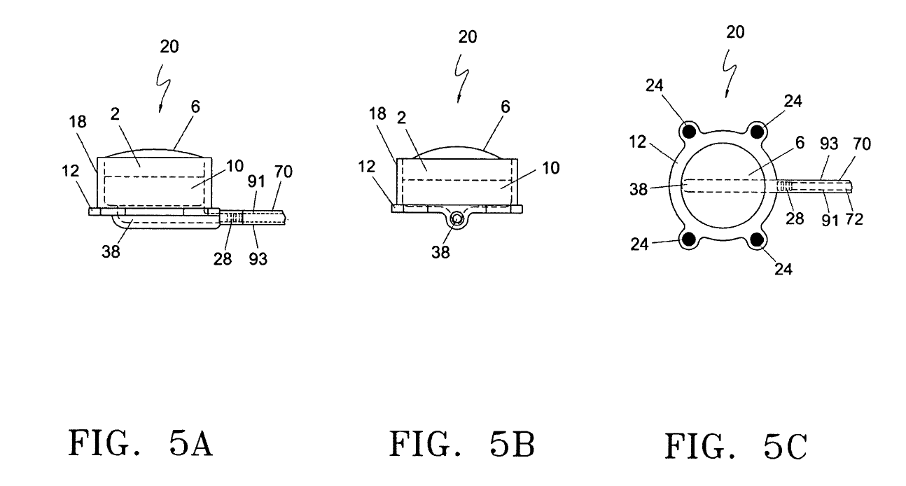

In one exemplary aspect, as illustrated in FIGS. 5A through 5C, the vascular access port 20 may define a trough 38 that forms an undulating portion in the bottom outer surface of the base 12 of the port 20 and extends below the reservoir 10 of the port 20. The trough 38 is fluidly connected to reservoir outlet stem 28 and is in fluid communication with port reservoir 10. In one aspect, trough 38 may extend substantially the entire width of the port reservoir 10, such as illustrated in FIGS. 5A through 5C. Alternatively, the trough 38 may extend a partial width of the port reservoir 10. In one aspect, the trough 38 is fluidly connected to the proximal portion of connection tube 70 at the stem 28 connection.

In another exemplary embodiment (not illustrated), the base 12 of the port 20 may have at least one protrusion that extends from the bottom surface of the base 12 to contact the bottom surface 3 of the interior cavity 61 when the port 20 is placed in the interior cavity 61 of the hub body 49. This enables the port 20 to have an enhanced sealing engagement with the bottom surface 3 of the interior cavity 61 of the hub 49 and to be better stabilized within the interior cavity 61 upon compression of the port 20 within the cavity 61. As the port 20 is compressed within the hub body 11, the protrusion allows the port 20 to be suspended within the interior cavity 61 of the port 20 between the extension tubes 50, 51 and clamps (not illustrated) on either side of the hub body 11 during the insert molding procedure. The space created between the bottom surface of the port 20 and the bottom surface 3 of the interior cavity 61 also allows for a sealing means, such as an adhesive or epoxy, to securely contact the bottom surface of the base 12 of the port 20 and the bottom surface 3 of the interior cavity 61 of the port 20. This feature may be used in either an insert-molded hub or a snap-fit hub. In another exemplary aspect, the port 20 and extension tubes 50, 51 may be pre-molded, and the resulting assembly may be placed inside of the interior cavity 61 of the hub body 11 before the entire catheter hub assembly 57 is insert molded.

After the port 20 and the hub body 11 are assembled, and the connection tube 70 is assembled to the port 20, the base 12 of port 20 is then secured to the bottom surface 3 of the bottom portion 34 of the interior cavity 61 of the hub 49. In one exemplary aspect, the base 12 of the port housing 18 is secured to the bottom surface 3 of the interior cavity 61 using various means such as, but not limited to, one or more of an adhesive, such as an expoxy, and/or a plurality of securement tabs 24, for example, those illustrated in FIGS. 3A-3D and 4A-4B. In one aspect (not illustrated), the tabs 24 may be defined therein the bottom surface 3 of the hub cavity 61 and/or connected to the bottom surface 3 and made of the same material as the bottom surface 3, such that the protrusions from the port body 20 may be securely placed over the tabs 24 to secure the port 20 inside of the hub body 11. The tabs 24 may then be ultrasonically heat-formed or welded together with the protrusions of the port 20. In one alternative aspect, an adhesive, such as an epoxy, may be applied to the bottom surface of the base 12 of the port 20 and/or the adhesive may be applied to the bottom of the tabs 24. The entire catheter hub assembly 57 may then be insert molded, which is a technique well known in the art.

The catheter shaft 7 is positioned within the opening 36 of the hub 49 to extend along the internal cavity 61 of the hub, and the extension tubes 50, 51 are placed within openings 32. The diameter of the openings 32, 36 which are defined in the hub body 11, and are configured to receive the extension tubes 50, 51 and the catheter shaft 7, may be varied to allow the finished hub 49 to accommodate different sized extension tubes 50, 51 and catheter shaft 7. As described above, and also illustrated in FIGS. 1A and 2A, extension tubes 50, 51 are disposed substantially longitudinally in the interior cavity 61 of the hub 49 along a longitudinal axis of the catheter hub 49 and do not overlap each other. Similarly, after the insert molding procedure, channels 42, 52 are disposed substantially longitudinally in the interior cavity 61 of the hub 49 along a longitudinal axis of the catheter hub 49 and do not overlap each other.

The distal portion of at least one of extension tubes 50, 51 becomes fluidly sealed to at least a portion of the proximal ends of channels 42, 52 in the interior cavity 61 of the hub body 11 at transition point 55, such that the finished hub assembly 57 is fused to a distal portion of the extension tubes 50, 51 and a proximal portion of the catheter shaft 7, and it forms a single, unified structure. Extension tubes 50, 51 and the channels 42, 52 are fluidly joined and defined in surrounding relationship to vascular access port 20, as illustrated in FIGS. 1A and 3D, for example. Alternatively, in one aspect, the extension tubes 50, 51 and channels 42, 52 may be positioned such that they are both located on one side of the port 20 and connection tube 70.

The catheter hub assembly 57 may also be assembled using a snap-fit technique, followed by a potting technique, known in the art, as illustrated in FIGS. 3A-3D and 4A-4B. In this aspect, the catheter hub 49 may be comprised of two separate pieces, a bottom portion 34 and a top portion 30, which may be snap-fitted together to form the hub 49. Both the bottom portion 34 and the top portion 30 have a body 11 with an exterior surface and partial openings 32, 36 defined in the exterior surface of the hub body 11 at the proximal end 1 and the distal end 4 of the hub 49, respectively. Bottom portion 34 has a bottom surface 3. Top portion 30 has a pre-formed opening 22, which is defined in the exterior surface of the hub body 11.

Before the bottom portion 34 and top portion 30 are snap-fit together, the base of the assembled port 20, with septum 2, is secured to the bottom surface 3 of the interior cavity 61 using various means such as, but not limited to, one or more of an adhesive and/or a plurality of securement tabs 24, illustrated in FIGS. 3A-3D and 4A-4B, for example, as described above. In one aspect an adhesive may be applied to the bottom surface of the base 12 of the port 20. In another aspect, a plurality of securement tabs 24 may secure the edges of the housing 12 to the bottom surface of the port 3. In one aspect, the plurality of securement tabs 24 may optionally be used to secure the base 12 of the port 20 to the bottom surface of the port 20.

In one aspect, the base of the port 20 may comprise a plurality of protrusions extending from the base 12 and defined therein the base 12 of the port 20. In one exemplary aspect, the base 12 may comprise four protrusions that are defined therein the base and extend from the base 12 of the port 20, spaced equidistantly from one another along the base 12 of the port 20, such as illustrated in FIGS. 4B and 5C. The protrusions may have pre-formed openings (not illustrated) in the center of each protrusion, through which a plurality of tabs 24 may be inserted and which allow for sealing engagement between the tabs 24 and the bottom surface 3 of the interior cavity 61.

In one aspect, the bottom surface of the tabs 24 may contact the bottom surface 3 of the interior cavity 61 and are secured to the bottom surface 3 of the interior cavity 61 using an adhesive, such as an epoxy. In one aspect, the top surface of tabs 24 may have a lip (not illustrated) which extends from the tab 24 inside the pre-formed opening, extends outwardly, and overhangs the surrounding base 12 of the port 20, so as to further enhance the engagement between the tab 24, the bottom surface 3 of the interior cavity 61, and the base of the port 20. In one aspect, as illustrated in FIG. 3A, the bottom portion 34 is pre-assembled with a plurality of extension tube securement tabs 26 that are defined in sealing engagement with the bottom surface 3 of the interior cavity 61. A plurality of tabs 26 function as securement means for the extension tubes 50, 51 therein the hub body 11. In one aspect, tabs 26 may be connected to the bottom surface 3 of the interior cavity 61 of the hub 49 or may also be incorporated into the bottom surface 3 of the interior cavity 61, similar to the plurality of tabs 24, described above.

As illustrated in FIG. 3C, after the lower portion 34 and upper portion 30 are joined together, they mate along a parting line 37 that is defined by a horizontal axis that bisects the catheter hub 49. In one aspect, the top and bottom portions 34, 30 may be sealed using any feasible sealing engagement mechanism that is well known in the art and suitable for sealing the top and bottom portions together. In the assembled state, the hub body 11 defines a plurality of openings 32, 36 in the exterior surface of the hub body 11. In one aspect, as described above, at least one opening 32 is defined therein the proximal end 1 of the body. In one exemplary aspect, two openings 32 are defined in the proximal end 1 of the hub body 49 and are configured for receiving the distal portion of extension tubes 50, 51. In one exemplary aspect, at least one opening 36 is defined in the distal end 4 of the hub body 11 and is configured for receiving the proximal portion of catheter shaft 7.

In one aspect, the bottom portion 34 of the hub body 11 is illustrated in FIG. 3D, after the port 20 has been sealed into the bottom surface 3 of the interior cavity 61. The extension tubes 50, 51 enter the catheter hub 49 through openings 32 and extend substantially longitudinally along a longitudinal axis of the hub 11 in the hub cavity 61 to transition point 58, where lumens 66, 67 of extension tubes 50, 51 are in sealed, fluid communication with lumens 9 and 19 of the catheter shaft 7, respectively. In one aspect, the extension tubes 50 and 51 may be held in place by extension tube securement tabs 26 at the proximal end 1 of the hub body 11. The tabs 26 function as a means for securing the extension tubes 50, 51 within the hub body 11. In one exemplary aspect, at least one extension tube securement tab 26 is located on each side of the extension tubes 50, 51. Extension tubes 50, 51 are fluidly connected to lumens 9, 19 of the catheter shaft 7 at transition point 58 at the distal end 4 of the hub body 11. In alternative embodiments, extension tubes 50, 51 may both be positioned on the same side of the port 20, instead of on opposite sides of the port 20.

In one aspect, FIG. 6 illustrates an exemplary method of infusing an infusate from a vascular access catheter comprising catheter hub assembly 57 into a patient's blood vessel. One skilled in the art will appreciate that the method of injecting an infusate using the catheter hub assembly 57 may encompass the use of any of the embodiments of the catheter hub assembly 57 described herein and illustrated in FIGS. 1 through 6. In one aspect, the method comprises providing any suitable catheter comprising the catheter hub assembly 57 described in any of FIGS. 1 through 6 and inserting a needle into surface 6 of the septum 2 of the port 20 and into the reservoir 10. Optionally, at least a portion of a guidewire may be inserted to access the third infusion lumen 27, if necessary. This process involves inserting at least a portion of a guidewire into the needle 23, through the reservoir, into a lumen 72 of the connection tube 70, and further into the third lumen 27 of the catheter shaft 7, advancing the guidewire through the third/infusion lumen 27 and into a vessel in a patient body; inserting the catheter into a vessel in a patient body over the guidewire; positioning the distal portion of the catheter at a desired location within the target vessel; and removing the guidewire from the catheter hub assembly 57 of the catheter.

In one aspect, any suitable needle 23 may be used with the vascular access port 20, such as, but not limited to, a non-coring Huber style needle. The Huber needle has a deflected point with an opening facing to the side of the needle shaft (i.e., a “beveled” needle design) which prevents the needle from coring out material from the silicone septum. This type of needle also helps to prevent permanent leaks or the infusion of cored silicone emboli into the patient. The needle 23 may be removably inserted into at least a portion of the surface 6 of septum 2 which is located exterior to the exterior surface 11 of the catheter hub body 11, and into the reservoir 10 to deliver drugs and other therapies and for blood sampling, as described below. The self-sealing septum 2 may be punctured up to several thousand times with needle 23 and still be able to be resealed into the port reservoir 10.

The method further comprises providing an infusate; and injecting the infusate through the needle 23, into the port 20, into the reservoir 10, into the connection tube 70, and into the third lumen 27 of the catheter, and into the body. In one aspect, the infusate may be selected from the group consisting of anti-restenosis agents, anti-thrombogenic agents, anti-inflammatory agents, anti-thrombotic agents, saline, contrast agents, urokinase, streptokinase, tissue plasminogen activator (t-PA), fibrinolytic agents, anti-proliferative agents, chemotherapeutics, and anti-coagulants.

In another aspect, a method for injecting infusates, including under high pressure for CT injection, is also provided. This method involves providing a vascular access catheter with catheter hub assembly 57, as described herein for use with a catheter, providing a contrast agent, and injecting the contrast agent under high pressure through the surface 6 of the septum 2 of the catheter hub assembly 57 into the third lumen 27 of the catheter and the port 20 and into the body for computed tomography.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. The words “including” and “having,” as used herein including the claims, shall have the same meaning as the word “comprising.” Those familiar with the art may recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the selected embodiments of the invention. Those skilled in the art may recognize other equivalents to the specific embodiments described herein which equivalents are intended to be encompassed by the claims attached hereto.

1. A catheter hub for use with a vascular access catheter, comprising:
a body having a proximal end, a distal end, an exterior surface, and defining an interior cavity having a bottom surface, wherein the exterior surface of the body further defines a plurality of openings, wherein at least one opening of the plurality of openings is defined therein the proximal end, and at least one opening of the plurality of openings is defined therein the distal end; and
a vascular access port positioned at least partially therein the interior cavity, wherein the vascular access port comprises:
a housing having a base and a wall extending therefrom the base; and
at least one self-sealing septum having an outer surface, the at least one septum being configured for a leak-proof connection to an upper portion of the wall of the housing, wherein a portion of a bottom surface of the at least one septum and portions of the wall and base of the housing define at least one reservoir, wherein a portion of the housing is in fluid communication with the at least one reservoir, and wherein at least a portion of the outer surface of the at least one septum is defined therein a port opening of the plurality of openings of the body.

2. The catheter hub of claim 1, wherein the base of the housing is positioned therein the interior cavity of the body substantially parallel to a horizontal plane bisecting the body.

3. The catheter hub of claim 1, wherein the base of the housing is mounted therein a portion of the interior cavity of the body.

4. The catheter hub of claim 1, wherein the base is sealingly engaged to a portion of the bottom surface of the interior cavity of the body.

5. The catheter hub of claim 1, wherein a portion of the housing defines a reservoir outlet stem that is in fluid communication with the at least one reservoir.

6. The catheter hub of claim 1, wherein the at least a portion of the outer surface of the at least one septum positioned therein the port opening is circumferentially surrounded by portions of the exterior surface of the hub body.

7. A vascular access catheter comprising a hub assembly, wherein the hub assembly comprises:
a body having a proximal end, a distal end, an exterior surface, and defining an interior cavity having a bottom surface, wherein the exterior surface of the body further defines at least one proximal opening defined therein the proximal end of the body, wherein the exterior surface of the body further defines at least one distal opening defined therein the distal end of the body that is configured to receive at least a portion of a proximal portion of a catheter shaft, wherein the body further defines at least one channel that is integral to the at least one distal opening;
a plurality of extension tubes and at least one connection tube, each extension tube and the at least one connection tube having a proximal end and a distal end, wherein each of the at least one proximal opening is configured to receive the distal end of one extension tube, and wherein, respectively, the distal end of one extension tube is fluidly connected to a proximal portion of one channel of the body; and
a vascular access port positioned at least partially therein the interior cavity, wherein the vascular access port comprises:
a housing having a base and a wall extending therefrom the base; and
at least one self-sealing septum having an outer surface, the at least one septum being mounted therein an upper portion of the wall of the housing, wherein a portion of a bottom surface of the septum and portions of the wall and base of the housing define at least one reservoir, wherein a portion of the housing is in fluid communication with the at least one reservoir, and wherein at least a portion of the outer surface of the at least one septum is defined therein a port opening that is defined therein the body.

8. The vascular access catheter of claim 7, wherein the vascular access catheter further comprises a catheter shaft comprising a plurality of lumens, each lumen having a proximal end and a distal end, and wherein at least a portion of the plurality of lumens of the catheter shaft are in fluid communication with the plurality of extension tubes and the at least one connection tube.

9. The vascular access catheter of claim 7, wherein the plurality of extension tubes of the catheter hub assembly comprises a first extension tube and a second extension tube, wherein the at least one channel comprises a first channel and a second channel, and wherein the distal end of the first extension tube is in fluid communication with the proximal portion of the first channel, and wherein the distal end of the second extension tube is in fluid communication with the proximal portion of the second channel.

10. The vascular access catheter of claim 8, wherein the plurality of lumens comprises a first lumen and a second lumen.

11. The vascular access catheter of claim 10, wherein the distal end of the first channel is in fluid communication with the proximal end of the first lumen, and wherein the distal end of the second channel is in fluid communication with the proximal end of the second lumen.

12. The vascular access catheter of claim 10, wherein the plurality of lumens further comprises a third lumen.

13. The vascular access catheter of claim 12, wherein the transverse cross-sectional area of the third lumen is smaller than the transverse cross-sectional area of the first lumen and the second lumen.

14. The vascular access catheter of claim 12, wherein the distal end of the at least one connection tube of the hub assembly is in fluid communication with at least a portion of the proximal end of the third lumen of the catheter shaft.

15. The vascular access catheter of claim 7, wherein a portion of the port housing of the hub assembly defines a reservoir outlet that is in fluid communication with the at least one reservoir.

16. The vascular access catheter of claim 15, wherein the reservoir outlet stem of the hub assembly further comprises a means for securing the connection tube to the reservoir outlet stem.

17. The vascular access catheter of claim 7, wherein at least a portion of the outer surface of the at least one septum of the access port positioned therein the port opening of the hub assembly is circumferentially surrounded by portions of the exterior surface of the body.

18. The vascular access catheter of claim 7, wherein the base of the housing of the access port of the hub assembly is positioned therein the interior cavity of the body substantially parallel to a horizontal plane bisecting the body.

19. The vascular access catheter of claim 7, wherein the base of the housing is mounted therein a portion of the interior cavity of the body.

20. The vascular access catheter of claim 7, wherein the base is sealingly engaged to a portion of the bottom surface of the interior cavity of the body.

21. The vascular access catheter of claim 7, wherein the bottom surface of the base of the access port of the hub assembly defines a trough that is fluidly connected to the reservoir outlet stem and is in fluid communication with the at least one reservoir.

22. The vascular access catheter of claim 9, wherein the first and second extension tubes and first and second channels are defined in surrounding relationship to the port of the hub assembly.

23. The vascular access catheter of claim 12, further comprising a liner, wherein the liner is positioned thereon at least a portion of an inner wall of the third lumen.

24. The vascular access catheter of claim 12, wherein the third lumen of the catheter shaft is configured to selectively receive at least a portion of a guidewire.

25. The vascular access catheter of claim 12, wherein the third lumen of the catheter shaft is in selective fluid communication with an infusate.

26. The vascular access catheter of claim 7, wherein the septum of the port of the hub assembly is configured to receive at least a portion of a needle.

27. The vascular access catheter of claim 7, wherein the body of the hub assembly is insert molded over the distal end of the plurality of extension tubes and the at least a portion of the proximal portion of the catheter shaft.

28. The vascular access catheter of claim 7, wherein the catheter is a hemodialysis catheter.

29. The vascular access catheter of claim 7, wherein the catheter is a peripherally inserted central catheter.

30. A method of infusing an infusate into a patient body, wherein the method comprises:
providing a vascular access catheter comprising a catheter shaft having a plurality of lumens, and a hub assembly, wherein the hub assembly comprises:
a body having a proximal end, a distal end, an exterior surface, and defining an interior cavity having a bottom surface, wherein the exterior surface of the body further defines at least one proximal opening defined therein the proximal end of the body, wherein the exterior surface of the body further defines at least one distal opening defined therein the distal end of the body that is configured to receive at least a portion of a proximal portion of a catheter shaft, wherein the body further defines at least one channel that is integral to the at least one distal opening;
a plurality of extension tubes and at least one connection tube, each extension tube and the connection tube having a proximal end and a distal end, wherein each of the at least one proximal opening is configured to receive the distal end of one extension tube, and wherein, respectively, the distal end of one extension tube is fluidly connected to a proximal portion of one channel of the body; and
a vascular access port positioned at least partially therein the interior cavity, wherein the vascular access port comprises:
a housing having a base and a wall extending therefrom the base; and
at least one self-sealing septum having an outer surface, the at least one septum being mounted therein an upper portion of the wall of the housing, wherein a portion of a bottom surface of the septum and portions of the wall and base of the housing define at least one reservoir, wherein a portion of the housing is in fluid communication with the at least one reservoir, and wherein at least a portion of the outer surface of the at least one septum is defined therein a port opening that is defined therein the body;
inserting a portion of the vascular access catheter into the patient's body;
providing an infusate; and
injecting the infusate through the outer surface of the septum into the reservoir, into one of the plurality of extension tubes, into one of the plurality of lumens of the catheter, and into the body.

31. The method of claim 30, wherein the catheter shaft further comprises a first lumen, a second lumen, and a third lumen, and the step of injecting further comprises injecting the infusate into the third lumen of the catheter.

32. The method of claim 30, wherein the catheter is a hemodialysis catheter.

33. The method of claim 30, wherein the catheter is a peripherally inserted central catheter.
